(19) United States
(12) Patent Application Publication
(10) Pub. No.: US 2004/0010307 A1
(43) Pub. Date: Jan. 15, 2004

(54) Implantable integral device and corresponding method for deflecting embolic material in blood flowing at an arterial bifurcation

(75) Inventors: Ygael Grad, Tel Aviv (IL); Nitzan Zafrir-Pachter, Karkur (IL); Avraham Rapaport, Tel Aviv (IL); Boaz Nishri, Doar Na Menashe (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: MindGuard Ltd.

(21) Appl. No.: 10/615,953

(22) Filed: Jul. 10, 2003

Related U.S. Application Data
(63) Continuation-in-part of application No. PCT/IL02/00022, filed on Jan. 11, 2002.
(63) Continuation-in-part of application No. 09/637,287, filed on Aug. 11, 2000.
Application No. 09/637,287 is a continuation-in-part of application No. 09/484,965, filed on Jan. 18, 2000, now Pat. No. 6,348,063.

(30) Foreign Application Priority Data
Jan. 11, 2001 (IL) 140869

(51) Int. Cl. A61F 2/06

(52) U.S. Cl. 623/1.15

(57) ABSTRACT

A device implantable in an artery at a bifurcation into a first branch supplying blood to a vital region having a high sensitivity to emboli in the blood, and a second branch supplying blood to a less vital region having a lower sensitivity to emboli in the blood. The implantable device is initially of a small diameter for facilitating its introduction into and deployment through the artery to the bifurcation, and is expandable to a larger diameter for implantation in the artery at the bifurcation. The implantable device includes a base element for anchoring in the artery at the bifurcation; and a deflector element for covering the inlet of the first branch at the bifurcation. The deflector element is formed with openings of a size and configuration to deflect emboli in the blood to the second branch without blocking blood flow through the second branch or through the first branch. The base element is a coil of tubular configuration having overlapping ends enabling it to be expanded from the initial diameter to the larger diameter. In the described preferred embodiments, the device is configured and dimensioned for implantation in the CCA at its bifurcation into the ICA and ECA.

### RELATED APPLICATIONS

The present application is a continuation-in-part of PCT/IL02/00022, filed Jan. 11, 2002, which claims priority from Israel Patent Application No. 140,869 filed Jan. 11, 2001. This application is also a continuation-in-part of U.S. patent application Ser. No. 09/637,287, filed Aug. 11, 2000, which is a continuation-in-part of U.S. patent application Ser. No. 09/484,965 filed Jan. 18, 2000, now U.S. Pat. No. 6,348,063.

### FIELD AND BACKGROUND OF THE INVENTION

The present invention relates to implantable devices for implanting in an artery of a patient at a bifurcation into a first branch supplying blood to a vital region having a high sensitivity to emboli in the blood, and a second branch supplying blood to a less vital region having a lower sensitivity to emboli in the blood. The invention is particularly useful for implantation in the common carotid artery (CCA) at its bifurcation into the internal carotid artery (ICA) and the external carotid artery (ECA), and is therefore described below with respect to this application.

A major portion of blood supply to the brain hemispheres is by two arteries in the neck, referred to as common carotid arteries (CCA), each of which branches off, or bifurcates, into an internal carotid artery (ICA) and an external carotid artery (ECA). Blood to the brain stem is supplied by two vertebral arteries.

Stroke is a leading cause of disability, death, and health care expenditure. It is the second most common cause of death worldwide, exceeded only by heart disease, and is the third most common cause of death in the U.S. as described in Heart and Stroke Statistical Update, Dallas, Tex., USA, American Heart Association, 2000.

Stroke is caused either by ischemia-infarction or intracranial hemorrhage. Infarction constitutes 85 to 90 percent of the total group in western countries, as described by Sacco, R. L., Tony, D., and Mohr, J. P., in Classification of ischemic Stroke, Stroke: Pathophysiology, Diagnosis and Management, Editors: Barnett, H. J. M., Mohr, J. P., Stein, B. M., and Yatsu, F.M., third edition, Churchill Livingtone, N.Y., USA, 1998, 271-83. The pathogenesis of ischemic stroke is complex with multiple potential mechanisms. The carotid plaque is only one source of stroke, accounting for no more than 15-20% of cases, as described by Petty, G. W., Brown, Jr., R. D., Whisnant, J. P., Sicks, J. D., O'Fallon, W. M., and, Wiebers, D. O., in Ischemic Stroke Subtypes, A Population-based Study of Incidence and Risk Factors, Stroke, 1999, 30, 2513-16. More frequently, infarcts are caused by more proximal sources of emboli, that is, the heart and the aortic arch. The commonest causes of cardioembolic stroke are nonrheumatic (often called nonvalvular) atrial fibrillation, prosthetic valves, rheumatic heart disease (RHD), congestive heart failure, and ischemic cardiomyopathy.

A recent population based study from Rochester, Minn., USA, found that the main identifiable subtype of ischemic stroke was cardioembolic with nearly 30% of cases, while all large vessel cervical and intracranial atherosclerosis with stenosis altogether constituted about 16% as described by Petty et al., ibib. Further, often multiple mechanisms, co-exist, as described by Caplan, L. R., in Multiple Potential Risks for Stroke JAMA 2000, 283, 1479-80. Wilson, R. G. and Jamieson, D. G., in Coexistence of Cardiac and Aortic Sources of Embolization and High-Grade Stenosis and Occlusion of the Internal Carotid Artery, J. Stroke Cerebrovasc Dis., 2000, 9, 27-30, reviewed the experience of Petty et al. with patients who had high grade internal carotid artery stenosis or occlusion, and also had cardiac and aortic evaluation. Potential cardiac or aortic sources of emboli were present in 54% of patients; aortic arch plaques greater than 4 mm in diameter were found in 26% of patients with severe internal carotid artery occlusive disease.

Prevention is clearly the most cost-effective approach to decreasing the burden of stroke. Available strategies to prevent stroke include medical treatment, surgery (carotid endarterectomy), and carotid stenting.

Current medical treatments include antiplatelet drugs, such as aspirin, ticlopidine, clopidogrel, and dipyridamol, for presumed athreothrombotic origin. These treatments reduce the risk for recurrent ischemic event by no more than 15-20%. Anticoagulants, such as Warfarin for non valvular atrial fibrilliation, reduce the risk by 60%; however, even in carefully conducted and monitored clinical trials, a substantial number of patients stopped anticoagulation, as described by Hart, R. G., Benavente, O., McBride, R., and, Pearce, L. A. in Antithrombotic Therapy to Prevent Stroke in Patients with Atrial Fibrillation; A Meta-Analysis, Ann Intern Med., 1999, 131, 492-501.

Carotid endarterectomy was shown to be beneficial in selected cases of medium grade symtomatic, and also in asymptomatic carotid stenosis, by greater than 60%, whenever complication rates are kept low, as described by Chassin, M. R., in Appropriate Use of Carotid Endarterectomy (editorial), N. Engl., J. Med., 12998, 339, 1468-71. Nevertheless, a high proportion of recurrent stroke was not related to the large artery atherothrombotic disease, but to other causes including cardioembolism, as recently reported by the NASCET (North American Symptomatic Endarterectomy Trial) investigators, Barnett, J. J. M., Gunton, R. W., Eliasziw, M., et al., in Causes and Severity of Ischemic Stroke in Patients with Internal Carotid Artery Stenosis, JAMA, 2000, 283, 1429-36. In fact, strokes related to cardioembolism tended to be more severe. The population of patients with carotid stenosis in ‘real life’ often includes patients with severe cardiac disease, concomitant protruding aortic arch atheroma, atrial fibrillation, or congestive heart failure. The proportion of patients with such concomitant disease increases substantially in an elderly population. Thus, the risk of recurrent cardioembolic stroke, even in patients operated for carotid stenosis, is estimated to be substantially higher, as described by Barnett, H. J. M., et al., ibid.

Carotid artery stenting has potential advantages of offering treatment to high risk patients with carotid stenosis, lowering peri-procedural risk, decreasing costs, and reducing patient inconvenience and discomfort. Preliminary results from clinical trials comparing carotid stenting to carotid endarterectomy have shown similar results, as described in Major Ongoing Stroke Trials, Stroke, 2000, 31, 557-2.

The approach to prevention of such a multi factorial complex syndrome as stroke is necessarily multifaceted. Carotid angioplasty, with stenting by itself, does not address additional sources of emboli, even after successful reduction of local stenosis. More efficient endovascular approaches to stroke prevention needs to take into account this complexity in cerebrovascular disease. In this context, an intravascular implant that also addresses prevention of emboli from proximal sources can be a valuable addition in the arsenal of the practicing physician.

Introducing filtering means into blood vessels, particularly into veins, has been known for some time. However, filtering devices known in the art are designed for filtering blood flowing in the vena cava, and for stopping embolic material having a diameter of the order of centimeters, but, are unsuitable to deal with arterial embolic material, with which the present invention is concerned, especially in cases where the diameter of such material is typically of the order of down to microns. Furthermore, the flow of blood in the veins does not resemble arterial flow by its hemodynamic properties. However, when considering the possible cerebral effects of even fine embolic material occluding an artery supplying blood to the brain, the consequences may cause irreversible brain damage, or, may even be fatal.

In light of the short period of time during which brain tissue can survive without blood supply, there is significant importance to providing suitable means for preventing even small sized embolic material from entering the internal carotid artery, so as to prevent brain damage, or, even death.

The size of the filaments that make up the deflecting and filtering element, and the Porosity Index thereof, defined hereinafter, are major features of the deflecting device of the present invention, as explained herein, below. By contrast, in venous blood filters currently known in the art, no particular attention has been given to the size of the filaments. It is noted that embolic material in venous blood is made up of only blood clots, while in arterial blood, it is necessary to deal with emboli featuring different materials, such as blood clots and atherosclerotic plaque debris, etc. Accordingly in order to provide efficient filtering means, a blood deflecting and filtering element should be of fine mesh. However, a fine mesh blood filter has a higher tendency toward occlusion.

It is also to be noted that the flow ratio between the ICA and the ECA is about 3:1-4:1. This flow ratio indicates the significantly high probability of embolic material flowing into the ICA rather than into the ECA. However, the ECA is a relatively non-hazardous artery because it supplies blood to superficial organs in the face and head, which are not life supporting and which receive blood supply from collateral blood vessels. Therefore, embolic material reaching these organs does not cause substantial damage to a subject.

The above-cited related patent application Ser. Nos. 09/637,287 and 09/484,965 (the latter having issued as U.S. Pat. No. 6,348,063), as well as PCT Application PCT/IL00/00145 (patent application Ser. No.09/950,027) discloses implantable devices implantable in an artery at a bifurcation into a first branch supplying blood to a vital region having a high sensitivity to emboli in the blood, and a second branch supplying blood to a less vital region having a lower sensitivity to emboli in the blood, for deflecting emboli in the blood to the second branch without blocking blood flow through the second branch or through the first branch. Such implantable devices were described particularly for implantation in the CCA to deflect emboli in the blood to the ECA without blocking blood flow through the ECA or through the ICA.

### OBJECT AND BRIEF SUMMARY OF THE INVENTION

An object of the present invention is to provide, in an implantable device of the type described in the above-cited patent applications, improvements which simplify the construction, reduce the cost of manufacture, and/or increase the flexibility of the device for internal positioning.

According to one aspect of the present invention, there is provided an implantable device implantable in an artery of a patient at a bifurcation thereof into a first branch supplying blood to a vital region having a high sensitivity to emboli in the blood, and a second branch supplying blood to a less vital region having a lower sensitivity to emboli in the blood; the implantable device being of tubular configuration initially of a small diameter for facilitating its introduction into and deployment through the artery to the bifurcation, and expandable to a larger diameter for implantation in the artery at the bifurcation; the implantable device comprising: a base element configured and dimensioned for anchoring the implantation device in the artery at the bifurcation; and a deflector element configured and dimensioned for covering the inlet of the first branch at the bifurcation when the implantable device is implanted in the artery; the deflector element being formed with openings therethrough of a size and configuration to deflect emboli in the blood to the second branch without blocking blood flow through the second branch or through the first branch; the base element being a coil of tubular configuration having overlapping ends in the initial diameter enabling it to be expanded from the initial diameter to the larger diameter.

According to further features in the described preferred embodiments, the coil is a perforated sheet coiled into the tubular configuration.

In one described preferred embodiment, the perforated sheet is dimensioned also to have overlapping ends when expanded to the larger diameter; in a second described embodiment, the perforated sheet is dimensioned to define a gap between its ends when expanded to the larger diameter. In both of the above described preferred embodiments, the deflector element is integrally formed with the perforated sheet, and the perforated sheet is formed with larger size openings than those of the deflector element.

According to further features in one described preferred embodiment, the perforated sheet of the base element is formed with a relatively stiff frame around its periphery and is dimensioned such that, when initially coiled into the tubular configuration, it has an initial small diameter of 1-4 mm and an expanded larger diameter of 5-30 mm.

In the described preferred embodiments, the perforated sheet of the base element, and the deflector element, are both of a braided material. Preferably, the perforated sheet is constructed of wires having a diameter of 100-1500 microns, were preferably 100-200 microns; and the deflector element is constructed of wires having a diameter of 20-75 microns. Preferably, the perforated sheet includes at least one radiographic opaque marker.

In the preferred embodiments of the invention described herein, the implantable device is configured and dimensioned for implantation in a patient's CCA at its bifurcation into the ICA (constituting the first branch) and the ECA (constituting the second branch) in order to reduce the risk of a stroke.

Further features and advantages of the invention will be apparent from the description below.

### BRIEF DESCRIPTION OF THE DRAWINGS

The invention is herein described, by way of example only, with reference to the accompanying drawings, wherein:

FIG. 1A is a schematic diagram illustrating a sheet of perforated material used for constructing a first exemplary preferred embodiment of implantable device in accordance with the present invention;

FIG. 1B is a schematic diagram illustrating a perforated sheet used for constructing a second exemplary preferred embodiment of implantable device in accordance with the present invention;

FIG. 2A is a schematic diagram illustrating a perspective view of an implantable device constructed from the sheet of perforated material of FIG. 1A, in accordance with the present invention;

FIG. 2B is a schematic diagram illustrating a perspective view of an implantable device constructed from the sheet of perforated material of FIG. 1B in accordance with the present invention;

FIG. 3A is a schematic diagram illustrating a cross sectional view of the implantable device of FIG. 2A or FIG. 2B, in its small-diameter form prior to deployment in accordance with the present invention;

FIG. 3B is a schematic diagram illustrating a cross sectional view of the implantable device of FIG. 2A or FIG. 2B in a first operative position during its deployment in accordance with the present invention;

FIG. 3C is a schematic diagram illustrating a cross sectional view of the implantable device of FIG. 2A or FIG. 2B in a second operative position during its deployment in accordance with the present invention;

FIG. 4A is a schematic diagram illustrating a side view of the implantable device of FIG. 2A or FIG. 2B in its contracted state during its deployment in accordance with the present invention;

FIG. 4B is a schematic diagram illustrating a side view of the implantable device of FIG. 2A or FIG. 2B in its partially expanded state during its deployment in accordance with the present invention;

FIG. 4C is a schematic diagram illustrating a side view of the implantable device of FIG. 2A or FIG. 2B in its fully expanded state after its deployment in accordance with the present invention;

FIG. 5A is a schematic diagram illustrating a side view of the implantable device of FIG. 2A or FIG. 2B deployed in the bifurcation of the carotid artery in accordance with the present invention; and

FIG. 5B is a schematic diagram illustrating a cross section view corresponding to the A-A plane in the side view of FIG. 5A, of the implantable device deployed in the carotid artery.

It is to be understood that the foregoing drawings, and the description below, are provided primarily for purposes of facilitating understanding the conceptual aspects of the invention and various possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

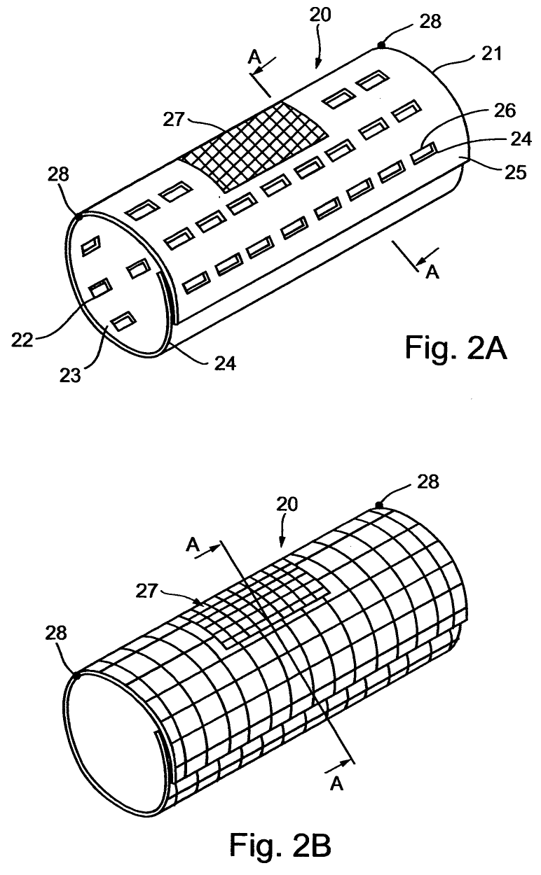

Deflecting device 20 illustrated in FIG. 2A is constructed from a base element in the form of a sheet of perforated material coiled into a tubular configuration so as to constitute a coil element 21. Coil element 21 has overlapping ends in its initial small diameter, enabling it to be expanded from its initial small diameter to a larger diameter for deployment, as will be described more particularly below. For simplification purposes, the detailed structure of deflecting device 20, and the elements and components thereof such as coil element 21, are not shown to scale.

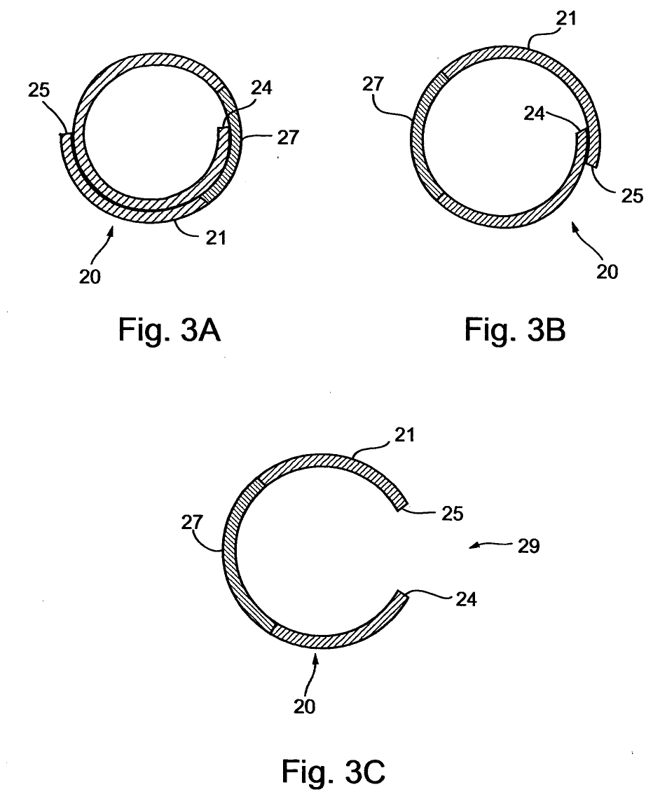

As shown in FIG. 2A, the sheet material defining coil element 21 is formed with perforations or apertures 22 and preferably with smooth edges 23. This permits the sheet to be coiled into a tubular configuration to constitute coil element 21 with one end 24 overlapping with the other end 25. In this embodiment as seen in FIG. 2A, end portion 24 is visible through aperture 26 in end portion 25. This embodiment is further illustrated in FIG. 3B, which is a cross sectional view of FIG. 2A, taken along the A-A plane.

As also shown in FIG. 2A, a portion 27 of the perforated sheet material used for making coil element 21, and integral with such sheet material, is constructed to serve as a deflector element. As will be described more particularly below, deflector element 27 is formed with openings therethrough of a size and configuration such that, when the coil element 21 is implanted within the bifurcation of the artery, the deflector element 27 deflects emboli in the blood to one branch (ECA) without blocking blood flow through the other branch (ICA) or through the one branch (ECA).

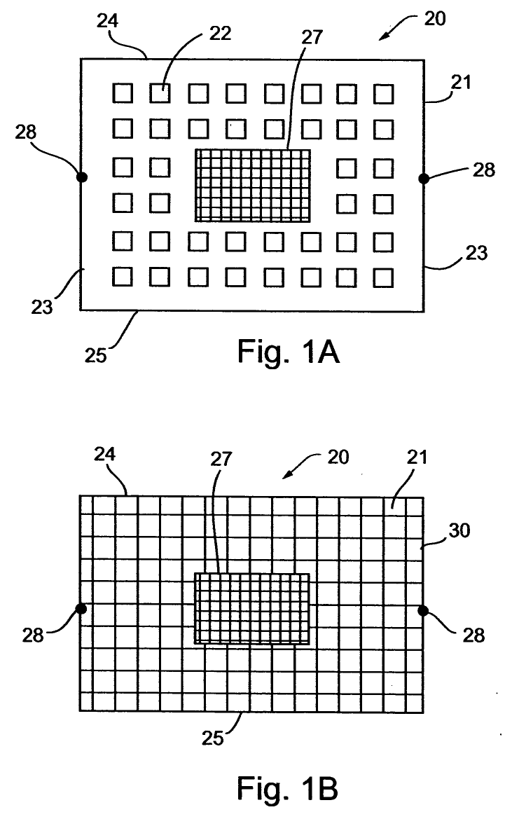

FIG. 1A is a schematic diagram illustrating the sheet of perforated material used for constructing the expandable dual diameter coil element 21 of deflecting device 20 illustrated in FIG. 2A. The perforations or apertures 22 permit growth of cells from the arterial walls onto the surface of coil element 21 of deflecting device 20, so as to firmly fix deflecting device 20 thereto and to prevent pathological damage to the arterial walls.

FIG. 1B is a schematic diagram illustrating an outer frame 30 surrounding the perimeter of a meshed structure used for constructing a second exemplary preferred embodiment of expandable dual diameter coil element 21 of deflecting device 20 illustrated in FIG. 2B. The meshed structure and its mesh dimensions may be of any suitable type, shape and sizes, for example, as used in conventional coronary stents.

In a preferred embodiment of the present invention, the perforated sheet defining the meshed structure of coil element 21 of FIG. 1B is made of braided material. The technique of flat braiding is well known in the art and is not described here. In another embodiment of the invention, it is possible to construct deflecting device 20 from a sheet of braided material. In this case, the flat braid is coiled into a deflecting device similar to that shown in FIG. 2B. In yet another embodiment of the present invention, the flat braid is manufactured from a shaped memory alloy, which can be formed into a cylindrical shape and processed to retain the desired shape.

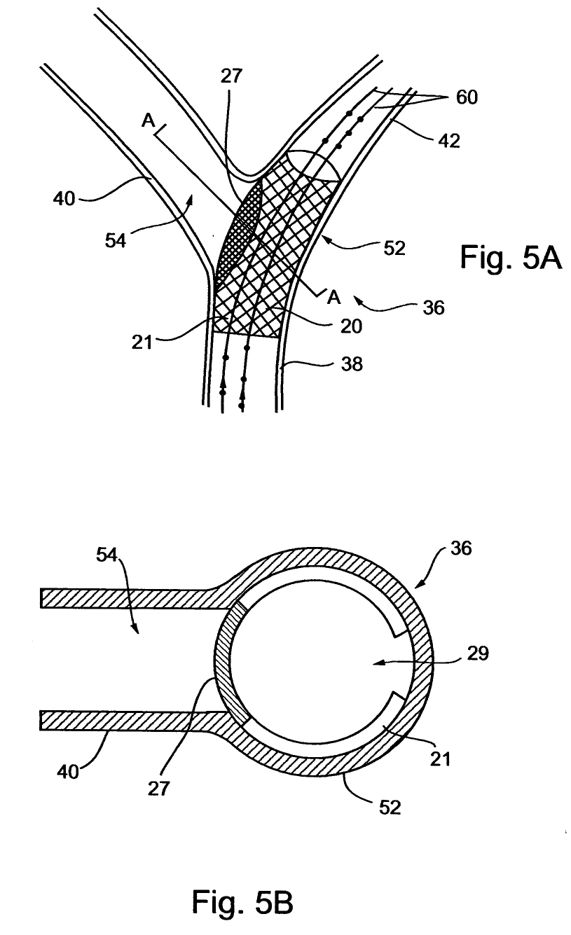

In deflecting device 20, a portion 27 of the perforated sheet material (FIGS. 1A and 2A) or framed mesh structure (FIGS. 1B and 2B), is either replaced by, or supplemented with, a substantially equivalently sized portion of a finely meshed zone to define deflector element 27, the physical requirements from which will be further described below. This finely meshed zone is the zone that, when deflecting device 20 is coiled and introduced into an artery, is positioned in front of aperture 54 of junction 52 (FIG. 5A). The actual shape, size, and, dimensions, of deflector element 27 is such that it covers the entire aperture or inlet 54 to internal carotid artery (ICA) 40 of carotid arterial bifurcation zone 52 (FIG. 5A).

Deflector element 27, integral with coil element 21 of deflecting device 20, has mesh size openings preferably in a range of between about 100 μm to about 700 μm, and, more preferably, in a range of between about 100 μm to about 400 μm, in order to effectively prevent an undesirable amount of dangerous embolic material flowing in the blood, from entering the internal carotid artery (ICA 40, FIG. 5A) in the region of an arterial bifurcation (arterial bifurcation zone 52, FIG. 5A). In general, in deflecting device 20, the open area of perforations or apertures 22 of the perforated sheet material as the first exemplary preferred embodiment of coil element 21 (FIGS. 1A and 2A), and, the open area or mesh size of the meshed structure of the second exemplary preferred embodiment of coil element 21 (FIGS. 1B and 2B), are significantly larger than the mesh size openings of deflector element 27 of coil element 21 required for deflecting the embolic material in the blood flowing at the arterial bifurcation.

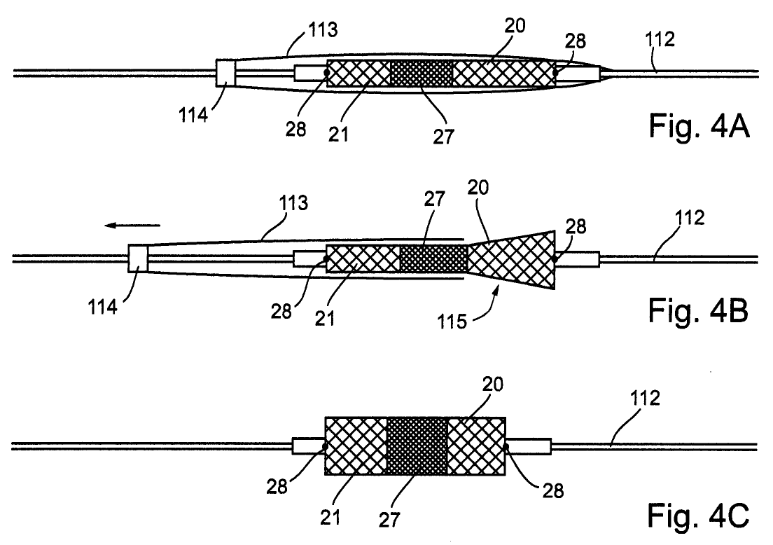

Deflecting device 20 is made of a material having an elasticity suitable for expanding from a contracted position in which it is deployed through the vasculator of a subject for preventing and/or treating a condition associated with embolic material in blood flowing at an arterial bifurcation, and expanded by means well known in the art, as will be further explained hereinafter with reference to FIGS. 4A through 4C.

Deflecting device 20 is schematically shown in FIG. 3A in the coiled construction in which it is deployed. In the condition depicted in FIG. 3A, dual diameter coil element 21 of deflecting device 20 is fully coiled or contracted, so that its initial diameter is substantially smaller than its expanded diameter. In the initial small-diameter condition, its overlapping end portions 24 and 25 do not necessarily need to be close to one another, and may be far apart, as shown in FIG. 3A.

FIG. 3B is a schematic diagram illustrating a cross section view of the deflecting device 21 of FIG. 2A or FIG. 2B, in a partially open or, expanded state, constituting a first operative position of deployment. In this partially open first operative state, opposing end portions 24 and 25 of coil element 21 also overlap.

FIG. 3C illustrates a preferred embodiment of the present invention in which the diameter of the blood vessel (for example, common carotid artery 38, as shown in FIG. 5A) where the deflecting device 20 is to be deployed, is relatively larger such that the end portions 24 and 25 of deflecting device 20, in its fully expanded state, do not overlap at all, and a gap 29 is formed between them. This situation is permissible as long as gap 29 lies against a wall of a blood vessel in which it is placed, and not against an opening to another blood vessel. This further illustrates the flexibility of expandable dual diameter coil element 21 of deflecting device 20 of the present invention, whereby deflecting device 20 can be used in conjunction with various blood vessel diameters, by automatically self-adjusting to unpredictable situations during deployment. Deployment of deflecting device 20 in this manner is preferred because the double wall resulting from overlapping of the end portions 24 and 25 of the coil element 21, which would otherwise reduce blood flow, is absent, thereby reducing stenosis.

Radio opaque markers 28 (for example, as shown in FIGS. 1A and 1B, in FIGS. 2A and 2B, and, in FIGS. 4A, 4B, and, 4C) are preferably provided and located at strategic positions, for example, on the perimeter, of coil element 21, which serve to aid a physician in the proper positioning of deflecting device 20 within an artery, especially within the region or vicinity of an arterial bifurcation in a subject having a condition associated with embolic material. Markers 28 are visible under radiographic equipment. Other markers can also be provided, according to known teachings in the art. For instance, markers 28 can be gold points that may be used to position coil element 21 of deflecting device 20 also with respect to rotation around a longitudinal axis of deflecting device 20.

Preferably, deflecting device 20 has an essentially cylindrical shape with its body such that its coil element 21 firmly contacts the inner walls of the external carotid artery and the common carotid artery at the arterial bifurcation. Such contact causes a growth of arterial wall into the net-like configuration of coil element 21, and strongly anchors deflecting device 20 in the artery, thus preventing its accidental displacement. The physiological processes leading to such anchoring are well known in the art, and are therefore not discussed herein in detail for the sake of brevity.

Introduction, insertion, and deployment of deflecting device 20, including its coil element 21 and its deflector element 27, are illustrated in FIGS. 4A-4C. Employing a self-expandable stroke preventing device at this location is more convenient in many cases because of the mobility of the subject's neck. This self-expandable feature and property provides for better anchoring of deflecting device 20 in the region of a bifurcation zone at an arterial junction of a subject.

FIG. 4A shows deflecting device 20 in its folded or contracted state; FIG. 4B shows it during the first stage of expansion; and FIG. 4C shows it in a fully expanded state. Deflecting device 20 is supported on a guide wire 112 which is used to introduce and guide deflecting device 20 to the arterial bifurcation. In the folded position or contracted state, deflecting device 20 is covered with an envelope 113, preferably made of polymeric material, for keeping deflecting device 20 in the folded or contracted state. Envelope 113 is connected to a retraction ring 114 which is pulled away from deflecting device 20 by a mechanism (not shown), well known in the art of stent deployment.

Referring now to FIG. 4B, when ring 114 is pulled away in the direction of the drawn arrow, envelope 113 is pulled away with it, uncovering a portion 115 of deflecting device 20. Since envelope 113 no longer constrains portion 115 to remain in the folded or contracted state, and since the normal operating position of deflecting device 20 is expanded, portion 115 starts expanding to its normally operative, expanded state. This process is completed, as shown in FIG. 4C, when envelope 113 has been completely removed, and deflecting device 20 is in its fully expanded position or state.

In the normally operative, expanded state, for example, as illustrated in FIG. 4C, radially directional elastic forces of the expandable property of expandable dual diameter coil element 21 operate to keep coil element 21, and therefore deflecting device 20, expanded, whereby deflecting device 20 is anchored in its location and is less susceptible to undesired displacement as compared to deployment of balloon expanded stents. Following completion and positioning of deflecting device 20, guide wire 112 is withdrawn from the subject, as in any other similar stent deployment procedure.

Other methods of deploying deflecting device 20 of the present invention are well known to a person having ordinary skill in the art. As another example, envelope 113 (FIGS. 4A and 4B) could be constructed by sewing a sleeve (not shown) using an open stitch. After positioning deflecting device 20 in the proper location, the end of such a thread, of which the sleeve is sewn, is pulled back fraying the material and allowing deflecting device 20 to expand, as shown in FIG. 4C.

FIG. 5A illustrates a carotid artery portion, generally designated 36, in which the common carotid artery (CCA) is designated 38, the internal carotid artery (ICA) is designated 40, and, the external carotid artery (ECA) is designated 42. Blood flowing throughout carotid artery portion 36 is indicated in FIG. 5A by the space between all other designated arteries and deflecting device elements and components.

Deflecting device 20 is positioned within arterial bifurcation zone 52, with deflector element 27 extending opposite inlet 54 of ICA 40. Coil element 21, functioning as an anchoring base for deflecting device 20, becomes firmly anchored in the inner walls of common carotid artery 38 and of external carotid artery 42, respectively, with deflector element 27 extending across inlet 54 of internal carotid artery 40. In this position, embolic material, which is schematically illustrated as particles in blood flowing along flow lines 60 in FIG. 5A, flows or moves with the flowing blood into common carotid artery 38. Upon contacting deflector element 27 as a result of the fluid motion of the blood, the embolic material particles are prevented from entering ICA 40 because the size of the particles is larger than the mesh size of deflector element 27, whereby, the embolic material particles are thus deflected into external carotid artery 42 of arterial bifurcation zone 52.

FIG. 5B is a cross-section view taken along the A-A plane of FIG. 5A. Referring to FIGS. 5A and 5B, the gap 29 results because the diameter of common carotid artery 38 is greater than the fully-expanded diameter of coil element 21. The importance here of radio opaque markers 28 (FIGS. 1, 2, and, 4) in the proper positioning of deflecting device 20 is clearly apparent. For instance, if at least part of gap 29 faces inlet 54 of internal carotid artery 40, rather than wall 52 of external carotid artery 42, the proper intended functionality of deflecting device 20 would be significantly limited.

As will be apparent to a person having ordinary skill in the art, expandable dual diameter coil element 21 does not necessarily need to be self-expandable; thus, coil element 21 of deflecting device 20 can be made of a non-self-expandable perforated sheet material (FIGS. 1A and 2A), or, of a meshed structure (FIGS. 1B and 2B) that is expandable under pressure supplied by a mechanism, such as by an implantable balloon, separate from, but, operative with, coil element 21. In this case, deployment of deflecting device 20 is carried out as for conventional stents by placing deflecting device 20 in a coiled position or contracted state around an expandable balloon, following by expanding the balloon under pressure after deflecting device 20 has reached the desired location. This is a conventional procedure and is, therefore, not illustrated in the figures, for the sake of brevity.

Deflecting device 20 of the present invention can be constructed in a way very similar to conventional stents. A person having ordinary skill in the art is knowledgeable of the various materials and methods suitable to make deflecting device 20 of the present invention. For instance, deflecting device 20 can be made of a material selected from the group consisting of nitinol, polymeric material, stainless steel, and combinations thereof.

Deflecting device 20 in the coiled position or contracted state can also be provided in a manner known to a person having ordinary skill in the art, for example, in a manner similar to that described in detail in PCT publication WO 99/48441 (U.S. Pat. No. 6,048,636), the contents of which are incorporated herein by reference, or, in any other suitable way.

Expandable dual diameter coil element 21 of deflecting device 20 of the present invention, can also be constructed using well known techniques of photochemical engraving, or, another etching process.

Preferably, deflector element 27 integral with coil element 21 of deflecting device 20, has openings in a range of between about 100 μm to about 700 μm, and, more preferably, in a range of between about 100 μm to about 400 μm, in order to effectively prevent an undesirable amount of dangerous embolic material flowing in the blood, from entering the internal carotid artery (ICA 40, FIG. 5A) in the region of an arterial bifurcation (arterial bifurcation zone 52, FIG. 5A). The diameters of expandable dual diameter coil element, and therefore of deflecting device 20 may somewhat vary, according to actual conditions associated with embolic material, of different subjects. The first diameter of coil element 21 of deflecting device 20 in the coiled position or contracted state varies, preferably, in the range of between about 1 mm to about 4 mm, and more preferably, in the range of between about 1 mm to about 3 mm. The second diameter of coil element 21 of deflecting device 20 in the open position or expanded state varies, preferably, in the range of between about 5 mm to about 35 mm, and more preferably, in the range of between about 5 mm to about 30 mm. The diameter of the wire making up the body or coil element 21 of deflecting device 20 is preferably in the range of between about 100 μm to about 1500 μm, and more preferably, in the range of between about 100 μm to about 200 μm. The diameter of the wire used for constructing deflecting and filtering element 27 is preferably in the range of between about 20 μm to about 75 μm, and more preferably, in the range of between about 20 μm to about 40 μm. Of course, the entire coil element 21, and, therefore, the entire deflecting device 20, can also be constructed using wire of the same dimensions as that of deflector element 27, whereby there would be no difference in mesh size between coil element 21 of deflecting device 20 and deflector element 27. In such case, a strengthening mechanism, for example ribs, may be required for proper performance during normal operation for treating a subject.

Deflector element 27 of deflecting device 20 preferably fulfills certain pre-determined conditions, several of which are described herein below. Various types of deflector elements 27, featuring different geometrical shapes, configurations, sizes, and, exhibiting desirable properties, may be constructed for fulfilling the following described conditions.

When testing deflecting device 20 under the following physiological conditions in the carotid region of a subject:

Reₐᵥ=200-500,

BPM (heart beats per minute)=40-180,

Womersley=2-7,

wherein Reₐᵥ is the average Reynolds number of the blood flowing at an arterial bifurcation of the carotid region, and, Womersley is the dimensionless heart beat parameter, the following conditions should preferably be met by deflecting and filtering element 27, of coil element 21 of deflecting device 20:

(1) Reprox is, preferably, in the range of between about 0.3 to about 30, and, more preferably, in the range of between 0 and about 4, and, is also preferably, equal to or less than 1, in accordance with creeping or Stokes' flow; and,

(2) Shear Stress is in the range of between less than about 100 dyne/cm² and greater than about 2 dyne/cm², wherein Reprox is the Reynolds number for a single wire of which deflecting and filtering element 27 is made, and, the shear stress is measured at deflecting device 20. As known to a person having ordinary skill in the art, the smaller Reₚᵣₒₓ is, the better the performance of deflecting device 20. However, deflecting device 20 may also operate at larger values of Reₚᵣₒₓ than indicated above, since the present invention is by no means limited to any specific value of Reₚᵣₒₓ.

Deflecting device 20 according to the present invention is useful in a variety of cases. Some illustrative indications are listed below:

(1) Embolic strokes from proximal sources. These are:

Atrial fibrillation (2.5 million in the U.S.A. in 1999);

Mechanical heart valve (225,000 procedures performed annually in the U.S.);

Subjects at high risk for recurrent embolism for a certain period (S.B.E.);

Subjects at high risk for proximal emboli and absolute contraindications for anticoagulation;

Subjects at high risk for proximal emboli failing best medical treatment.

(2) In cases in where carotid stents are introduced to treat local stenosis, it is possible to introduce and deploy the deflecting device of the present invention during the same procedure if there are concomitant high risk proximal sources of emboli. These are, for instance:

Protruding Aortic arch atheroma (more than ⅓ of symptomatic subjects);

Severe carotid stenosis with concomitant cardiac disease;

Severe carotid stenosis in subjects undergoing heart surgery (5% on the statistical basis of 600,000 coronary bypass surgeries).

The deflecting device may be combined with a conventional stent, for example, for the treatment of bifurcation lesions, where a stent is positioned in the side branch and the deflecting device in the main branch, wherein the conventional stent is deployed at the internal carotid artery and addresses local stenosis. The insertion and deployment techniques are similar to those employed in connection with a conventional stent. Bilateral procedures can be performed during the same session without increased risk thus enabling deployment of bilateral carotid divertors. Moreover, the deflector element of the deflecting device is similarly effective in deflecting embolic material above a certain size, irrespective of the composition of the embolic material. Given that embolic matter may be composed of thrombotic material, platelet-fibrin particles, cholesterol, atheroma, or, calcified particles, such a mechanical diversion or deflection has an inherent advantage of being general to any embolic composition.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

While the invention has been described in conjunction with specific embodiments and examples thereof, it is evident that many alternatives, modifications and variations will be apparent to those skilled in the art. Accordingly, it is intended to embrace all such alternatives, modifications and variations that fall within the spirit and scope of the appended claims.

What is claimed is:

1. An implantable device implantable in an artery of a patient at a bifurcation thereof into a first branch supplying blood to a vital region having a high sensitivity to emboli in the blood, and a second branch supplying blood to a less vital region having a lower sensitivity to emboli in the blood;
said implantable device being of tubular configuration initially of a small diameter for facilitating its introduction into and deployment through the artery to said bifurcation, and expandable to a larger diameter for implantation in the artery at said bifurcation;
said implantable device comprising: a base element configured and dimensioned for anchoring said implantation device in the artery at said bifurcation; and a deflector element configured and dimensioned for covering the inlet of said first branch at the bifurcation when the implantable device is implanted in said artery;
said deflector element being formed with openings therethrough of a size and configuration to deflect emboli in the blood to said second branch without blocking blood flow through said second branch or through said first branch;
said base element being a coil of tubular configuration having overlapping ends in said initial diameter enabling it to be expanded from said initial diameter to said larger diameter.

2. The implantable device according to claim 1, wherein said coil is a perforated sheet coiled into said tubular configuration.

3. The implantable device according to claim 2, wherein said perforated sheet is dimensioned also to have overlapping ends when expanded to said larger diameter.

4. The implantable device according to claim 2, wherein said perforated sheet is dimensioned to define a gap between its ends when expanded to said larger diameter.

5. The implantable device according to claim 2, wherein said deflector element is integrally formed with said perforated sheet.

6. The implantable device according to claim 2, wherein said perforated sheet of the base element is formed with larger size openings than those of said deflector element.

7. The implantable device according to claim 2, wherein said perforated sheet of the base element is formed with a relatively stiff frame around its periphery.

8. The implantable device according to claim 2, wherein said perforated sheet of the base element is dimensioned such that, when initially coiled into said tubular configuration, it has an initial small diameter of 1-4 mm.

9. The implantable device according to claim 8, wherein said perforated sheet of the base element is dimensioned such that its expanded larger diameter is 5-30 mm.

10. The implantable device according to claim 2, wherein said perforated sheet of the base element and said deflector element are both of a braided material.

11. The implantable device according to claim 2, wherein said perforated sheet of the base element is constructed of wires having a diameter of 100-1500 μm.

12. The implantable device according to claim 2, wherein said perforated sheet of the base element is constructed of wires having a diameter of 100-200 μm.

13. The implantable device according to claim 2, wherein said deflector element is constructed of wires having a diameter of 20-75 μm.

14. The implantable device according to claim 2, wherein said perforated sheet of the base element includes at least one radiographic opaque marker.

15. The implantable device according to claim 1, wherein the implantable device is configured and dimensioned for implantation in a patient's CCA at its bifurcation into the ICA constituting said first branch, and the ECA constituting said second branch.

16. An implantable device for implantation in a patient's CCA at its bifurcation into the ICA and the ECA, said implantable device being of tubular configuration initially of small diameter and expandable to a large diameter when implanted;
said implantable device comprising: a base element configured and dimensioned for anchoring the implantable device in said CCA, and a deflector element configured and dimensioned for covering the inlet of said ICA;
said deflector element being formed with openings therethrough of a size and configuration to deflect emboli in the blood to said ECA without blocking blood flow through said ECA or through said ICA;
said base element including a coil of tubular configuration having overlapping ends in said initial diameter enabling it to be expanded from said initial diameter to said larger diameter.

17. The implantable device according to claim 16, wherein said coil is a perforated sheet coiled into said tubular configuration and having overlapping ends enabling it to be expanded to said larger diameter.

18. The implantable device according to claim 17, wherein said deflector element is integrally formed with said perforated sheet.

19. The implantable device according to claim 17, wherein said perforated sheet is dimensioned also to have overlapping ends when expanded to said larger diameter.

20. The implantable device according to claim 17, wherein said perforated sheet is dimensioned to define a gap between its ends when expanded to said larger diameter.
